(19) United States
(12) Patent Application Publication
(10) Pub. No.: US 2005/0060783 A1
(43) Pub. Date: Mar. 24, 2005

(54) High strength, impact resistant knee protector

(76) Inventors: Jerome Gold, Virginia Beach, VA (US); Peter Krajian, Ridgefield, NJ (US)

Correspondence Address: FOLEY AND LARDNER;SUITE 500, 3000 K STREET NW, WASHINGTON, DC, 20007, US

(21) Appl. No.: 10/900,406

(22) Filed: Jul. 28, 2004

Related U.S. Application Data
(63) Continuation of application No. 10/684,824, filed on Oct. 14, 2003, now Pat. No. 6,839,906.
Application No. 10/684,824 is a continuation-in-part of application No. 10/230,118, filed on Aug. 29, 2002, now abandoned.

(51) Int. Cl. A61F 5/00; A41D 13/06

(52) U.S. Cl. 2/24

(57) ABSTRACT

A high strength, impact resistant knee protector that absorbs and dissipates energy from high impact contact includes a pre-formed high strength, lightweight material contoured to the knee. The high strength, lightweight material is angled to sufficiently cover about 50%-75% of the circumference of the knee area. At least one layer of impact resistant foam padding is secured to a side of the pre-formed high strength, lightweight material. The knee protector includes an outer cover having elastomeric qualities.

### CROSS-REFERENCE TO RELATED PATENT APPLICATIONS

This application is a continuation of U.S. application Ser. No. 10/684,824, now U.S. Pat. No. ______ filed Oct. 14, 2003, which is a continuation-in-part of application Ser. No. 10/230,118, filed Aug. 29, 2002. The contents of the prior applications are incorporated herein by reference.

### BACKGROUND OF INVENTION

A high strength knee protector that withstands extreme high impact force is provided. The purpose in creating the high strength impact resistant knee protector is to provide ultimate protection to the knee area against severe trauma. Throughout sports history, the most common and feared injury in contact sports in particular has been injury to the knee area and its major ligaments, such as the anterior cruciate ligament (ACL) and meniscus ligaments, due to trauma to this extremely sensitive area of the knee joint. The much exposed ACL ligament runs alongside the outer section of the knee. This ligament is extremely sensitive and, because of its location, extremely vulnerable to injury. Throughout the history of modem sports knee injuries have resulted in the premature ending of careers of many prominent athletes. The kneepads athletes wear today offer minimal protection due to material make up and design. In addition the braces offered today provide minimal protection against unnatural and awkward movements of the knee joint. Contemporary braces keep the knee stabilized but, because of their complex design, offer the athlete minimal lateral movement, a maneuver crucial to many sports. In many instances, the braces are uncomfortable to wear, causing athletes to forgo the braces in game situations.

Thus, there is a need for a high strength, impact resistant knee protector that is comfortable to wear and does not hinder maneuverability.

### SUMMARY

In one embodiment, there is provided a high strength, impact resistant knee protector that absorbs and dissipates energy from high impact contact. The knee protector includes a pre-formed, high strength, lightweight material contoured to the knee. The high strength, lightweight material has been bent by machine and angled to sufficiently cover 50% to 75% of the circumference of the knee area. The high strength, lightweight material is then covered with a foam padding of similar type material. An elastic sleeve is sewn to form a pocket for receiving the material and padding. The thickness gauge of the high strength lightweight material is such that it offers strength and lightness of weight. The thickness of the padding material surrounding the high strength, lightweight material should be sufficient to absorb impact and cushion the knee when wearing the high strength impact resistant knee protector. When contact is made to the outermost layer of the knee protector, the energy from the strike travels rearward from the outermost foam padding material and then is dissipated and absorbed by the high strength, lightweight material. In a preferred embodiment, the foam materials for the foam layers comprise a high density tight cell foam material or similar type material.

In another embodiment, a high strength impact resistant knee protector includes a single piece of preformed high strength, lightweight material that is contoured about the knee between 50% to 75% of the circumference thereof; at least one layer of impact resistant foam padding is secured onto a side of the preformed piece; and an outer cover having elastomeric qualities forms a pocket that receives the preformed piece.

There also is provided a high strength, impact resistant knee protector including a single piece of pre-formed high strength, lightweight material that is contoured about the knee between 50% to 75% of the circumference thereof; two layers of impact resistant foam padding, wherein one of the layers is secured to the top of the pre-formed piece and one of the layers is secured to the bottom of the pre-formed piece; and an outer cover having elastomeric qualities that forms a pocket that receives the pre-formed piece. In preferred embodiments, the high strength, lightweight material is titanium, aluminum, stainless steel, hard plastic or similar impact material. Titanium is the most preferred high strength, lightweight material.

In yet another embodiment, a method of making a knee protector includes contouring a piece of high strength, lightweight material to cover about 50% to 75% of a circumference of a knee area; securing a layer of impact resistant foam padding to at least one side of the contoured piece; sewing an elastomeric outer cover to form a pocket; and receiving the contoured piece in the pocket to form a high strength, impact resistant knee protector.

### BRIEF DESCRIPTION OF THE DRAWINGS

FIG. 1 provides a schematic, from a side view, of an exemplary knee protector.

FIG. 2 provides a cross sectional view of an exemplary knee protector.

FIG. 3 shows a schematic, from a top view, of an exemplary knee protector.

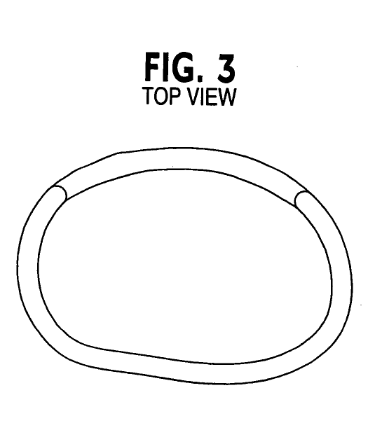

### DETAILED DESCRIPTION

A high strength, impact resistant knee protector is provided that absorbs and dissipates energy from high impact contact. The knee protector includes a pre-formed high strength, lightweight material contoured to the knee. The high strength, lightweight material is angled to sufficiently cover about 50%-75% of the circumference of the knee area. At least one layer of impact resistant foam padding is secured to a side of the pre-formed high strength, lightweight material. The knee protector includes an outer cover having elastomeric qualities.

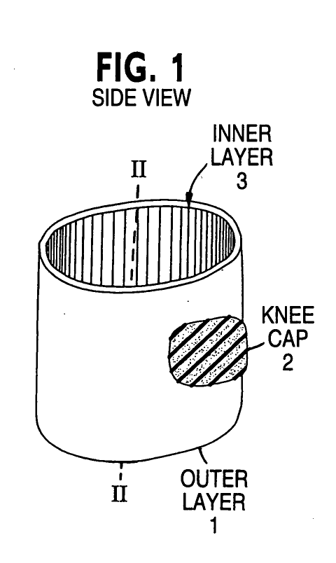

Referring now to the drawings, FIG. 1 shows one embodiment of a high strength, impact resistant knee protector. In the embodiment shown, the knee protector consists of an elastic outer layer (1) which is comprised of a nylon or similar type material. A knee cap cushion (2) is sewn onto the surface of the elastic outer layer and acts as an initial high impact absorber that dissipates energy prior to the impact energy reaching the pre-formed high strength, lightweight material.

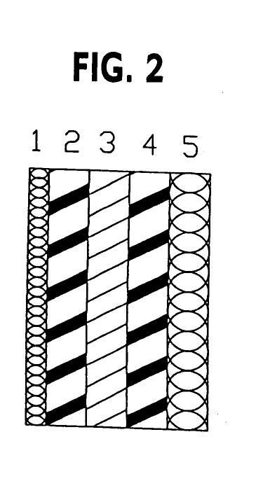

The elastic outer layer is sewn to form a pocket for receiving the material and padding, which will be described hereinafter. The elastic inner layer (3) is consistent with the outer layer so as to form a sleeve which would slide on and off the leg, and to hug the knee. The elastic inner layer consists of a nylon material or similar type material thereof. FIG. 2 allows a view of the materials utilized to form the knee protector. Layers 1 and 5, as depicted in FIG. 2, represent the front and back layers of the knee protector, which covers the entire inner and outer sections of the knee where major ligaments are much exposed.

The outer and inner covers (1-5) consist of an elastic material or similar type material thereof. The padding consists of two layers (2, 4) of padding or foam type material thereof. The tubular cover of foam type material (2, 4) is utilized to dissipate impact energy received from the outer layer (1, 5) before reaching the inner layer. A pre-formed lightweight material (3) contoured to fit the knee is utilized to provide impact protection to the joint and to give strength to the protector. When formed and contoured, the high strength, lightweight material protects both sides of the knee, so that between 50%-75% of the circumference of the knee is covered. The high strength, lightweight material acts as a shock absorber to dissipate impact energy received from the outer layers. Foam type material padding (2, 4) is then adhesively bonded to the high strength lightweight material (3) using a high strength bonding adhesive and is machine stitched to form a pocket surrounding the lightweight material which is completely enclosed.

1. A high strength, impact resistant knee protector comprising:
a single piece of pre-formed high strength, lightweight material that is contoured about the knee between 50% to 75% of the circumference thereof;
two layers of impact resistant foam padding, wherein one of said layers is secured to the top of said pre-formed piece and one of said layers is secured to the bottom of said pre-formed piece; and
an outer cover having elastomeric qualities that forms a pocket that receives said pre-formed piece.

2. The knee protector of claim 1, wherein the high strength, lightweight material is titanium, aluminum, stainless steel, hard plastic or similar impact material.

3. The knee protector of claim 1, wherein the high strength, lightweight material is titanium.

4. A method of making a knee protector, comprising:
contouring a piece of high strength, lightweight material to cover about 50% to 75% of a circumference of a knee area;
securing a layer of impact resistant foam padding to at least one side of said contoured piece;
sewing an elastomeric outer cover to form a pocket; and
receiving said contoured piece in said pocket to form a high strength, impact resistant knee protector.

5. The method of claim 4, wherein the contouring step includes contouring titanium, aluminum, stainless steel, hard plastic or similar impact material.

6. The method of claim 4, wherein the contouring step includes contouring titanium.
